(19) United States
(12) Patent Application Publication
(10) Pub. No.: US 2025/0345048 A1
(43) Pub. Date: Nov. 13, 2025

(54) CATHETER FOR USE WITH PERCUTANEOUS VALVE LEAFLET RESECTIONS

(71) Applicant: Hussam Fawzi Ahmad Hawamdeh, Ft. Smith, AR (US)

(72) Inventor: Hussam Fawzi Ahmad Hawamdeh, Ft. Smith, AR (US)

(21) Appl. No.: 19/080,805

(22) Filed: Mar. 15, 2025

Related U.S. Application Data
(60) Provisional application No. 63/644,725, filed on May 9, 2024.

(51) Int. Cl. A61B 17/00; A61B 17/12

(52) U.S. Cl. CPC A61B 17/00234; A61B 17/12109; A61B 17/12131; A61B 2017/00243; A61B 2017/00305; A61B 2017/00477; A61B 2017/00862; A61B 2017/00867; A61B 2017/00876; A61B 2017/00929; A61B 2017/00938; A61B 2017/1205

(57) ABSTRACT

A heart catheter for removing at least one leaflet of a defective heart valve includes a catheter sheath having a proximal end, a distal end, and an interior passageway extending between the distal end and the proximal end. A catheter wire is insertable into the passageway of the catheter sheath, with the catheter wire having a proximal end and a distal end. A first basket is coupled to the distal end of the catheter wire and is sized and configured for being received interiorly of a heart valve. A second basket is coupled to the distal end of the catheter wire and is disposed distally (more aortic) than the first basket. The second basket is sized and configured to interiorly receive one or more valve leaflets and the first basket.

### CROSS-REFERENCE TO RELATED APPLICATIONS

This application claims the priority of U.S. Provisional Patent Application No. 64/644,725 filed 9 May 2024

### TECHNICAL FIELD OF THE INVENTION

The present invention relates to medical devices and, in particular, to medical catheters that are used in connection with performing procedures on hearts such as heart valve percutaneous valve leaflet resections.

### BACKGROUND OF THE INVENTION

The human heart includes a series of valves that control the flow of blood between the various parts of the heart, and between the heart interior, and the heart exterior and, in particular, through the aorta to the rest of the of the body. These valves include the tricuspid valve, the mitral valve, the pulmonary valve, and the aortic valve.

The tricuspid valve includes three leaflets, and controls the flow of blood between the right atrium and the right ventricle. The pulmonary valve is a three-leaflet valve and controls the flow of blood between the right ventricle and the lungs (pulmonary artery).

The mitral valve is a two-leaflet containing valve, and controls the flow of blood between the left atrium and the left ventricle. Finally, the aortic valve is a three-leaflet valve (which in some occasion can have only two leaflets) and controls the flow of blood between the left ventricle and the aorta, which is the primary artery through which blood leaves the heart and is ultimately distributed through the vast circulatory network of arteries, capillaries, and veins to all parts of the body.

Unfortunately, these valves do not always last for a lifetime, but rather need to be replaced if they wear out or become damaged.

Heart valve replacement is a fairly common procedure, since heart valves can wear out over time, or otherwise are malformed from birth. Both native heart valves and replacement heart valves become worn out and need to be replaced.

Typically, when the native heart valve (the original valve that is native to the heart) wears out, it is replaced by a replacement valve. When a native valve is replaced, the native valve is resected, and the first-generation replacement valve is sewn onto the aortic anulus to replace the old valve using open heart surgery and with the use of heart-lung bypass machine while the heart is completely stopped.

Hopefully, the patient will do well with the replacement valve. Unfortunately, first-generation replacement valves (which are actually the second-generation of valve in the heart after the native valve) also have a propensity to wear out and need to be replaced.

The current technology employed in replacing first-generation replacement valves with second-generation replacement valves includes inserting the second-generation replacement valve over the first-generation replacement valve, and leaving the first-generation replacement valve in place. Although this technique of having two valves does work well, room for improvement exists.

One area where room for improvement exists is that the second-generation replacement valve may not perfectly match the size, shape, and configuration of the first replacement valve. If such a mismatch occurs, the operational efficiency of the valve can be impacted adversely, a condition that is known as “patient prosthesis mismatch”. A second problem that results is that the dual-layer valve restricts blood flow through the valve when compared to the blood flow that would be permitted if the valve was properly sized for the opening. This occurs because the second-generation replacement valve is usually placed over the first replacement valve, and the second-generation replacement valve is usually smaller than the first-generation replacement valve.

Because the second-generation valve is smaller than the first-generation valve, the net result is that the valve opening is smaller, which thereby restricts the flow of blood, when compared to the flow of blood that would occur if the second-generation replacement valve permitted a valve opening that was as large as the valve opening permitted by the native heart valve.

Therefore, one objective of the present invention is to allow for complete resection of the native heart valve to allow for placement of a first generation replacement valve without the need for an open heart surgery that requires cardiac arrest and the use of heart-lung bypass machine, a second object of the present invention is to provide the implantation of a second-generation valve that does not require the second-generation valve to be placed over another valve.

### SUMMARY OF THE INVENTION

In accordance with the present invention, a heart catheter is provided for removing the soft part of a defective heart valve also known as valve leaflets. The heart catheter comprises a catheter sheath having a proximal end, a distal end, and an interior passageway extending between the distal end and proximal end. A catheter wire is insertable into the passageway of the catheter sheath and includes a proximal end and a distal end.

A first basket is coupled to the distal end of the catheter wire and is sized and configured for being received interiorly of a heart valve. The second basket has a hollow center through which the wire catheter passes. It is coupled to a distal end of a smaller catheter that allows it to move freely from the first basket and the catheter wire as well. It is free to be advanced or pulled back in relation to the outer layer of the catheter. The second basket is sized and configured to interiorly receive one or more valve leaflets and the first basket.

The first and second baskets are movable with respect to each other along the catheter wire between a disengaged position, wherein the first basket is separated from the second basket, and an engaged position wherein the first basket is nested within the second basket.

In a further preferred embodiment, the first basket has an interior surface and an exterior surface, and the second basket has an exterior surface and an interior surface. The first basket is configured to be received interiorly of the heart valve, and the second and first baskets are movable with respect to each other to capture at least one leaflet of the heart valve between the exterior surface of the first basket, and the interior surface of the second basket.

Preferably, the first basket has at least a portion that is comprised of an electrically conductive material capable of severing a defective heart valve leaflet from a heart using electrical cauterization (this is called electro-surgery).

These and other features of the present invention will become apparent to those skilled in the art. upon a review of the drawings and detailed description set forth below.

### BRIEF DESCRIPTION OF DRAWINGS

FIG. 1 is an exploded view of the heart catheter of the present invention;

FIG. 2 is a side view of the first basket of the present invention;

FIG. 3 is a sectional view taken along lines 3-3 of FIG. 2;

FIG. 4 is an enlarged view of a small portion of the wire mesh of the first basket of the present invention;

FIG. 5 is a side view of the second basket of the present invention;

FIG. 6 is a top view of the second basket of the present invention; (second basket also has a mesh or Gore-Tex wrap);

FIG. 7 is a side-sectional, schematic view of the first and second baskets of the present invention, being contained within the interior of the catheter sheath, adjacent to the distal end of the catheter sheath, it being understood that the shape shown was simplified for purpose of clarity elastration. (or pictures will need to be edited to reflect the cones with arches);

FIG. 7A is a sectional view of the proximal end (outside the body) of the catheter sheath and catheter of the present invention;

FIGS. 8-13 are progressive views of the catheter of the present invention resecting at least one leaflet from a defective heart valve; wherein,

FIG. 8 is a side sectional view that shows a dilator that is inserted in the catheter sheath, and is used to help dilate the artery, to facilitate the passage of the catheter sheet in the artery and into the left ventricle.

FIG. 8A is a side sectional view that shows a dilator that is inserted in the catheter sheath interiorly to the valve within the left ventricle that is interior to the valve;

FIG. 9 is a side sectional view showing the first and second baskets contained within a catheter, wherein the distal end of the catheter, and the first and second baskets are disposed interiorly of the valve;

FIG. 10 is a side sectional view of the catheter of the present invention showing the first basket being disposed interiorly of the valve, and the second basket being disposed exteriorly of the valve, with both of the first and second baskets being disposed exteriorly of the distal end of the catheter sheath, and wherein the first basket and second basket are placed in their disengaged position;

FIG. 11 is a side sectional view of the second basket and first basket moved into their engaged position, and wherein they have captured at least one valve leaflet between the exterior surface of the first basket and the interior surface of the second basket;

FIG. 12 is a side sectional view that shows the first basket being nested within the second basket, with the already resected valve leaflet being captured between the first basket and second basket;

FIG. 13 is a sectional view showing the first basket and second basket being nested together, with the resected valve leaflet therebetween, and wherein the first and second baskets have been moved to the interior passageway of the catheter sheath, so that they may ultimately be drawn through the catheter sheath and out the distal end of the catheter sheath;

FIG. 14 is an enlarged sectional view of a portion of the sectional view of FIG. 13, wherein the first and second baskets are nested together, with the at least one resected valve leaflet captured therein;

FIG. 15 is a sectional view taken similar to FIG. 13, showing a catheter having an alternative configuration;

FIG. 15A is a sectional view taken along lines 15A-15A showing the first basket and second basket being nested together, with the resected valve leaflet therebetween;

FIG. 16 is a schematic sectional view that shows the heart muscle in the diastolic phase (relaxed phase); and

FIG. 17 is a schematic sectional view that shows the heart muscle in the systolic phase (contracted phase).

### DETAILED DESCRIPTION

Turning now to the figures, a heart catheter 10 is shown that is provided for helping to resect one or more leaflets of a heart valve. As shown in FIG. 10, a heart valve 12 includes a first leaflet 14 and a second leaflet 16 and may include a third leaflet (not shown). As discussed above, one of the heart valves include first and second leaflets 14, 16, and the other three of the heart valves are tricuspid valves that contain three leaflets.

The valve 12 shown in the drawings is meant to depict the aortic valve. The aortic valve is a valve of the heart 18 that controls the flow of blood between an interior chamber of the heart, here shown as the left ventricle 20, and an exterior vessel of the heart 18, here shown as aorta 22. Blood is pumped from the interior of the left ventricle, through the aortic valve 12, and into the interior 24 of the aorta 22.

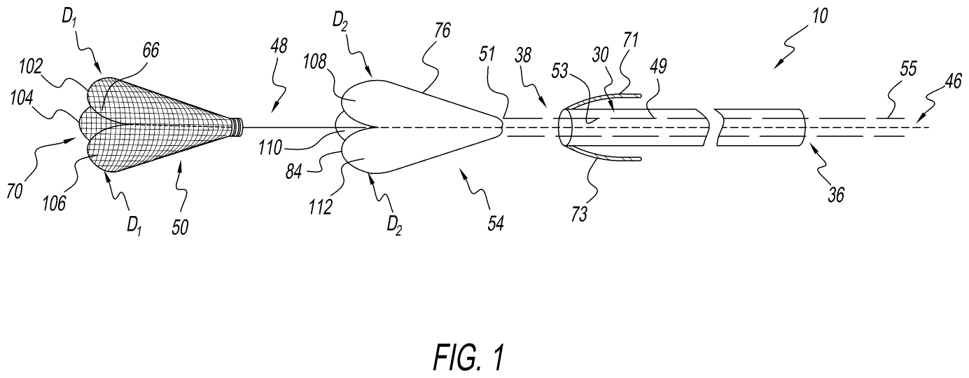

Returning to FIG. 1, the catheter 10 includes a catheter sheath 30, that generally comprises a hollow tube that is sized to fit within the interior of the arteries of a patient, so that a physician can maneuver the catheter 30 from a position exterior of the patient where the proximal end 36 of the catheter sheath 30 is disposed. From the proximal end 36, the doctor can manipulate tool-like devices, such as first and second basket 50, 54 which are disposed adjacent to the distal end 32 of the catheter sheath 30.

The catheter sheath 30 includes a hollow passageway 38 that extends generally between the proximal end 32 and distal end 36 so that both of the distal end 32 and proximal end 36 are open ends to thereby permit a catheter wire 42 that is inserted into the passageway 38 to simultaneously extend within the entire passageway 38, and still be disposed exteriorly of the proximal end 32 and distal end 36 of the catheter sheath.

As discussed above, the catheter wire 42 is disposed within the catheter sheath and has a length that is generally greater than the catheter sheath 30. The catheter wire 42 includes a proximal end 46 and a distal end 48. Controls (not shown) can be coupled to the proximal end 46 of the wire 42 to enable the user to manipulate tools, such as the first basket 50 and second basket 54 which are disposed adjacent to the distal end 48 of the catheter wire 42.

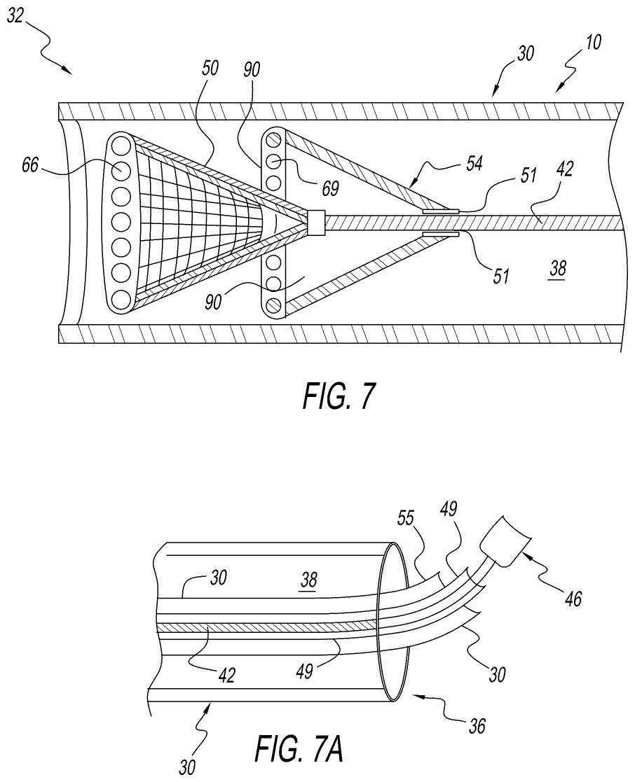

A catheter control cable/tube 49 has a distal end 51 (replaced the number on FIG. 7, it was marked as 92, changed it to 51) that is fixedly coupled to the second basket 54, a hollow interior 53 for slidably receiving the catheter wire 42, and a proximal end 55 which extends exteriorly of the tube 49. The catheter control cable 49 is semi-rigid and is provided for enabling the user to move the first basket 50 relative to, and independently of, the second basket 54.

A wide variety of controls can be employed, along with different types of catheter wires. For example, the catheter wire 42 can comprise or include fiberoptic cables that serve as a lens of a camera so that the user can have a visual display of what is occurring adjacent to the distal end 48 of the catheter wire. The catheter wire 42 may comprise a plurality of catheter wires that serve different functions.

For the sake of simplicity of view, only one catheter wire 42 is shown in the drawings, which is the catheter wire 42 by which the user can manipulate the first and second baskets 50, 54 and perform a procedure of severing one or more valve leaflets 14, 16 from a heart 18, which is the intended function of the catheter 10 of the present invention.

The first basket 50 is coupled to the distal end 48 of the catheter wire 42, and is sized and configured for being received interiorly of a valve, such as heart valve 12. A second valve basket 54 is coupled to the distal end 51 of the catheter tube 49 and is disposed proximally on the catheter wire 42 relative to the first basket 50. Catheter tube 49 has a hollow passage which catheter wire 42 runs-through, this configuration allows first basket 50 and second basket 54 to slide in relation to each other.

The second basket 54 external surface is preferably comprised of an impermeable material such as a Goretex® brand waterproof, breathable fabric membrane, which is generally impermeable to water and solids, and is available from W. L. Gore and Associates. Second basket 54 is sized and configured to interiorly receive one or more valve leaflets 14, 16 and the first basket 50.

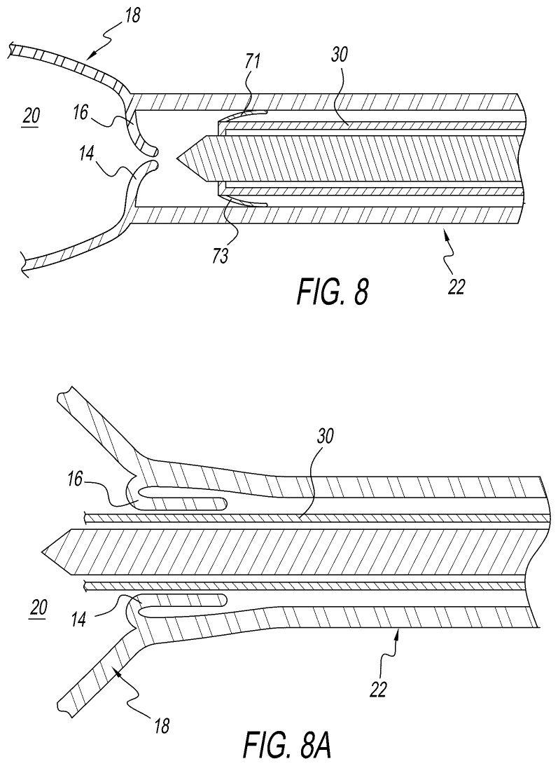

As will be disclosed in more detail with respect to FIGS. 8-14, the first basket 50 and second basket 54 are movable with respect to each other along the catheter wire 42 between a disengaged position as shown in FIG. 10, and an engaged position as shown in FIG. 14. In the disengaged position, the proximal end of the first basket 50 is disposed distally and exteriorly of the proximal end of the second basket 54.

The first and second baskets 50, 54 are movable into an engaged position, such as shown in FIG. 14, wherein the first basket 50 is nested within the interior of the second basket 54. In the engaged position, the rims of the first basket 50 and second basket 54 are magnetically coupled by the magnets placed on each of the first basket 50 and second basket 54, and are disposed adjacent to each other.

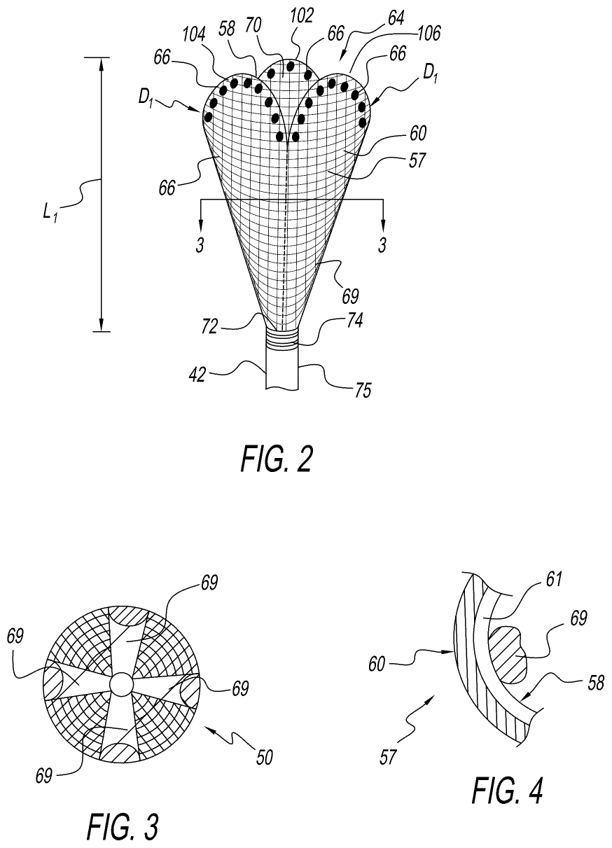

As best shown in FIGS. 1-4, the first basket 50 includes a distal rim 64 that is preferably made out of a nitinol material. The distal rim 64 should be flexible, but yet have a memory so that when the rim 64 is unencumbered by shape changing forces, the distal rim 64 should assume a generally annular configuration. A plurality of magnets 66 are disposed on the rim 64, in an array that comprises several magnets. The rim 64 has a diameter D1 that is generally similar to the diameter D2 of the second basket 54 rim.

The first basket 50 includes one or more generally axially extending support members that provide some structural rigidity, while still permitting the first basket 50 to have the flexibility to collapse, so that it may fit inside the relatively smaller diameter passageway of the catheter sheath 30.

The first basket 50 includes a generally mesh screen 57 that surrounds support members 69. The mesh screen 57 is preferably comprised of an electrically conductive material, such as wire.

As shown in FIG. 4, the mesh screen 57 includes an exterior surface 60 and an interior surface 58. The exterior surface 60 comprises exposed conductive material, such as aluminum, steel, copper, brash, titanium, and the like, which can convey electrical current.

A coating 61 is placed over the surface of the metal mesh on the interior 58 surface of the first basket 50. The coating 61 is preferably an electrically insulating coating, which prevents the exposed exterior surface 60 from shorting out when the device is being used to resect a valve leaflet by cauterizing the valve leaflet by running current through the metal mesh 60 which engages the leaflets 14, 16.

The first basket 50 includes a distal opening 70, and a closed proximal end 72. The shape of the basket 50 is preferably conically tapered, so that the diameter of the distal opening 70 is much greater than the diameter of the closed proximal end 72.

In this regard, the shape can generally be conical, frusto-conical, pyramidal, or frusto-pyramidal. A connector 74 is disposed at the extreme distal end 75 of the catheter wire 42 to fixedly couple and position the second basket 50 on the catheter wire 42.

The length L1 of the first basket 50 should be generally similar to the length L2 of the second basket 54, just as the diameter D1 of the first basket 50 should be generally similar to the diameter D2 of the second basket 54.

The rims 64, 76 of the respective first 50 and second 54 baskets should also be generally similar, so that the when the baskets 50, 54 are in the engaged position, the magnets 66 of the first basket 50 can be placed adjacent to, and in contact with, the magnets 84 on the rim 76 of the second basket 54 so the magnets can magnetically attach the first basket 50 to the second basket 54, to maintain the resected valve in a captured position between the exterior surface 60 of the first basket 50, and the interior surface 86 of the second basket 54.

Your attention is now directed to FIGS. 1, 2 and 10. You will notice that the first basket 50 and second basket 54 do not include circular rims. Rather, each of the first basket and second basket have segmented rims. The first basket 50 includes a first arched top segment 102, a second arched top segment 104, and a third arched top segment 106.

The second basket 54 has a first arched top segment 108, a second arched top segment 110, and a third arched top segment 112.

The purpose of the arched top segments is that they are shaped to better engage with the valves. In the drawings, the three-segment containing baskets are best employed with tricuspid valve leaflets, and the two-segment baskets are best employed with bicuspid valves.

As noted in in FIG. 1, arch-topped segments allow for a shape that mirrors the aortic valve architecture, which allow for complete resection of the valve intended to be resected (native or bioprosthetic valve)

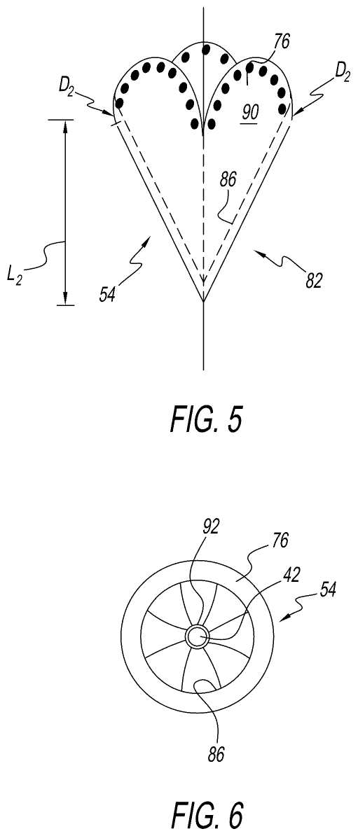

The second basket 54 is best shown in FIGS. 5-7. The second basket 54 includes an open distal end 58, and an open proximal end 77. The rim 76 is generally circular, and it is preferably made from a nitinol material so that the rim 76 has a memory, so that when the rim 76 is unencumbered by shape altering forces exerted on it, the rim 76 will assume a generally arched notch configuration.

As discussed above, a plurality of magnets 84 are arrayed along the rim 76, and are sized, configured, and positioned to be engaged with the magnets 66 of the first basket 50, when the first and second basket are in their engaged position.

The second basket 54 includes a body 82 that has an exterior surface. The second basket 54 also includes an open distal end 90, and an open proximal end 92. The proximal end 92 is open so that the catheter wire 42 may slide through the proximal end 92, to enable the first basket 50 to move with respect to the second basket 54.

This ability of the first basket 50 to move with respect to the second basket 54, enables the first and second baskets 50, 54 to move between a disengaged position, as shown in FIG. 10, and an engaged position as shown in FIG. 14. In the disengaged position (FIG. 10) the distal end 90 of the first basket 50 is actually separated from the rim 76 at the distal end 58 of the second basket 54, and the first basket 50 is not nested within the second basket 54 in an engaged position.

In the engaged position (FIG. 14) the first basket 50 is fully nested within the second basket 54, to capture and trap the leaflets 14, 16 between the exterior surface of the first basket 50 and the interior surface of the second basket 54. When in the engaged position, the plurality of magnets 66 of the first basket 50 are in magnetic contact, and coupled to the magnets 84 of the second basket 54. Through this magnetic contact the rims are attached together to thereby better prevent the leaflets 14, 16 that are trapped and captured between the first and second baskets 50, 54 from escaping capture.

Insofar as size, it is envisioned that the diameter of the catheter sheath 38 should be similar to the size of the sheath required for the valve that you are working with and the future valve that will be implanted after completion of valve resection. Typically, the valves, such as aortic valves, have a diameter of between about 19-36 mm, so that the diameter D1, D2 of the baskets at its widest part will also be somewhere between 19-36 mm.

The length of the baskets 50, 54 will primarily be somewhere around 20 mm. The First and second baskets 50, 54 should have complimentary shapes, so that they will nest well within each other.

If the first basket 50 is conically shaped, the second basket 54 should also be conically shaped. Similarly, if the first basket 50 is shaped as a pyramid, the second basket should also have a shape of a pyramid. Similarly, both of the baskets 50, 54 should be arch-topped, or both should be circular and planar. The skeleton members 69 should also be made of a nitinol material as is the rim.

The operation of the device 10 will now be discussed.

Turning first to FIG. 7, the catheter 10 is shown in a position where it is inserted into an artery, and is threaded through one or more arteries until it reaches the interior of the heart over a guiding wire, and is positioned in the left ventricle. It will be noted that the first basket 50 and second basket 54 are generally nested together, and are contained within the interior passageway 38 of the catheter sheath 30. They are both disposed near the distal end 32 of the catheter sheath 30, so that they can then be uncovered in the left ventricle as the catheter sheath 30 is being pulled out of the left ventricle and into the aorta, while the baskets remain in the ventricle.

Moving next to FIGS. 8 and 8A, a dilator member 70 is first inserted into the blood vessel 22 (over a guiding wire that extends into the left ventricle 20, not shown in drawing) and pushed to a position inside the left ventricle 20 of the heart as shown in the drawings. The purpose of the dilator 70 is to dilate the blood vessel 22 so that the catheter 30 can pass through the blood vessel 32, as well as, to allow for the delivery of a large catheter across a stenotic aortic valve 12.

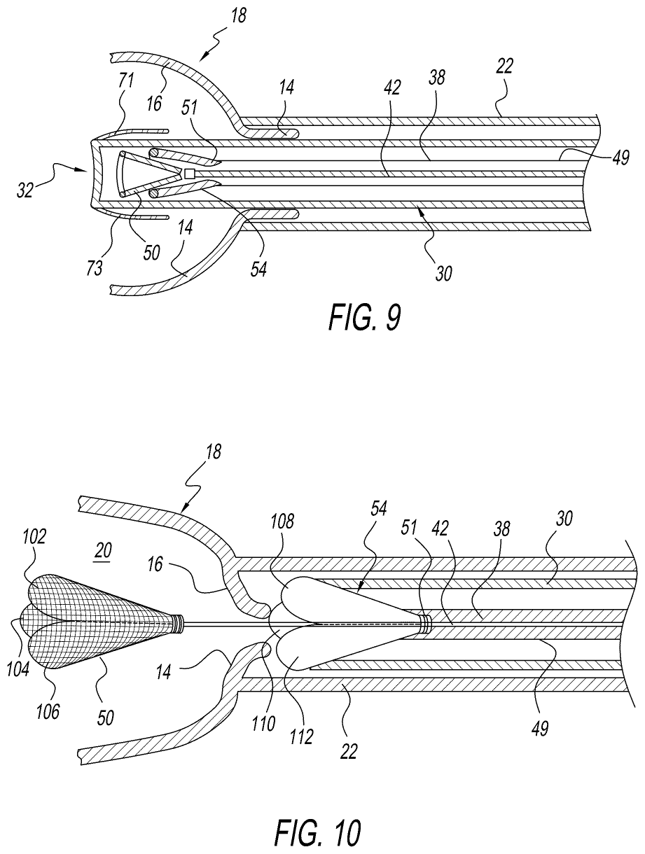

As shown in FIG. 9, the dilator 70 is removed from the catheter sheath 30, and the tool with the first 50 and second 54 basket and catheter wire 42 are inserted into the catheter sheath 30. The baskets 50, 54 are moved through the hollow passageway 38 until the first and second baskets 50, 54 are disposed adjacent to the distal end 32 of the catheter sheath 30, as shown in FIG. 9.

The distal end 32 of the catheter sheath 30 is shown being placed interiorly of the left ventricle 20, and interiorly of the heart valve 12, here shown as the aortic valve. In this position, the first and second baskets 50, 54 are contained within the catheter sheath 30.

The catheter sheath 30 will have an inner dilator that will help get the catheter sheath 30 to enter the ventricle by passing through the aortic valve with the least amount of resistance. Once the catheter sheath is within the interior of the left ventricle, the inner dilator will be removed. Once the dilator is removed, the first and second baskets 50, 54 will then be advanced into the interior passageway 38 of the catheter sheath 30.

The catheter sheath 30 is then moved distally, by the user, who controls the position of the catheter sheath by manipulating controls at the proximal end of the catheter sheath 30, or by manipulating the catheter sheath 30 itself. It will be noted that the distal end 32 of the catheter sheath 30 is inserted through the aortic valve, so that the distal end is positioned within the left ventricle 20 of the heart, and is disposed generally interiorly of the aortic valve 12 and its leaflets 14, 16.

At this point, both of the first basket 50 and second basket 54 are disposed within the left ventricle.

Turning now to FIG. 10, the catheter sheath 30 is retracted distally out of the left ventricle along with the second basket 54 which is also retracted so that it is disposed exteriorly of the aortic valve leaflets 14, 16. Meanwhile, the first basket 50 is maintained interiorly of the aortic valve 12, and is contained within the left ventricle 20. As shown in FIG. 10, the first and second baskets 50, 54 have been moved from their engaged position, as shown in FIGS. 7-9, to their disengaged position as shown in FIG. 10.

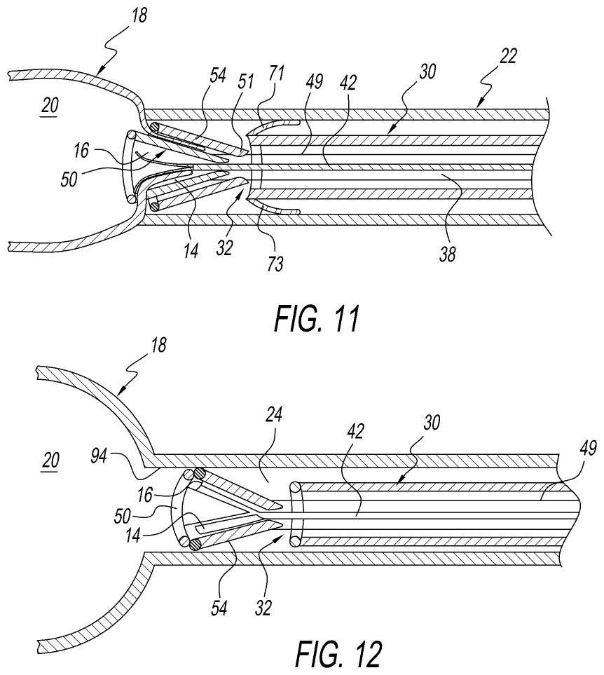

Turning next to FIG. 11, the catheter wire 42 is retracted to be moved in an aortic direction, so that the first basket 50 and second basket 54 are moved into an engaged position, wherein the first basket 50 is nested within the second basket 54. Captured between the exterior surface of the first basket 50 and the interior surface of the second basket 54 are the first and second leaflets 14, 16 of the aortic valve.

At this point, current is fed to the mesh body of the first basket 50 so that the exposed conductive mesh on the exterior surface of the first basket 50, can sever the leaflets 14, 16 from the heart 18, and so that the first and second leaflets 14, 16 can be fully captured between the first basket 50 and the second basket 54.

Although only first and second leaflets 14, 16 are shown, as the aortic valve is a tricuspid valve, it will be appreciated that preferably, the first leaflet 14, second leaflet 16, and third leaflet (not shown) are all captured between the first basket 50 exterior surface and the second basket 54 interior surface.

When the tissue of the leaflets 14, 16 has been severed, the first and second baskets 50, 54 remain in their engaged position and the baskets 50, 54 are pulled proximally away from the aortic anulus to which they were formerly attached. At this point, the aortic valve 12 has no remaining leaflets, but has a generally clean aortic anulus 94 to which a new valve can be attached.

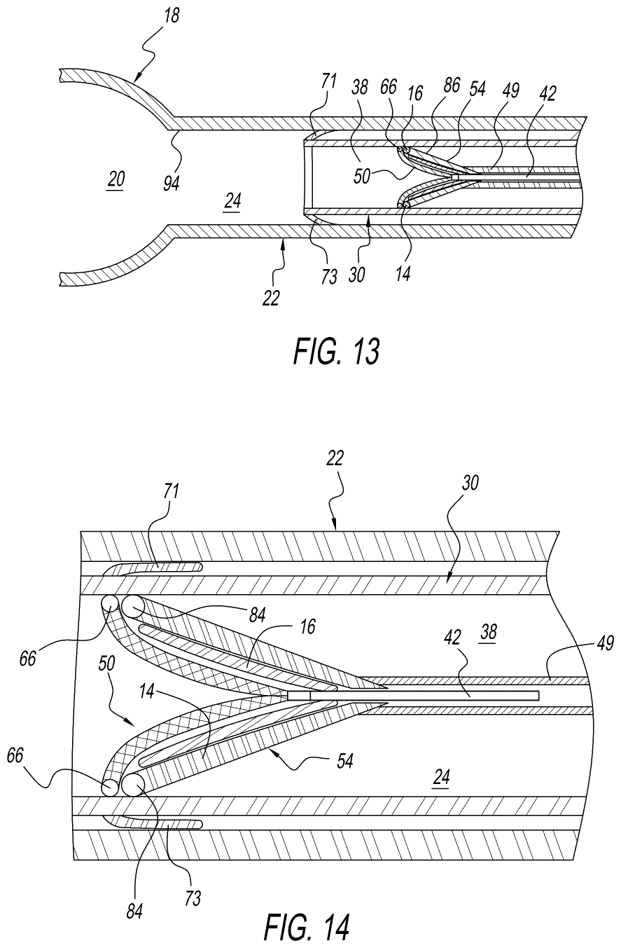

Moving on to FIGS. 13 and 14, the first and second baskets 50, 54 continue to be retracted proximally, through the action of the catheter wire 42, until the first and second baskets 50, 54 are contained within the catheter sheath 30. The first and second baskets 50, 54 continue to be retracted proximally in the engaged position (where the first and second baskets 50, 54 are nested together), until they are fully retracted, and are pulled out of the distal end of the catheter sheath and outside the body of the patient.

When this occurs, a new catheter (not shown) can be inserted into the interior 38 of the catheter sheath 30. This new catheter is preferably designed to transport the soon-to-be-implanted second-generation valve (prosthetic or bioprosthetic valve), by coupling it to the aortic anulus 94. When this occurs, and the new second-generation valve is coupled to the aortic anulus 94, the user will then have a working valve.

Because the new, second-generation valve (not shown) is not placed over the existing first-generation valve, the second-generation valve can be made as wide as the aortic anulus, which thereby can enable the new replacement valve to open wider than the valves of the prior art, to thereby allow more blood to flow out of the left ventricle, and into the aorta.

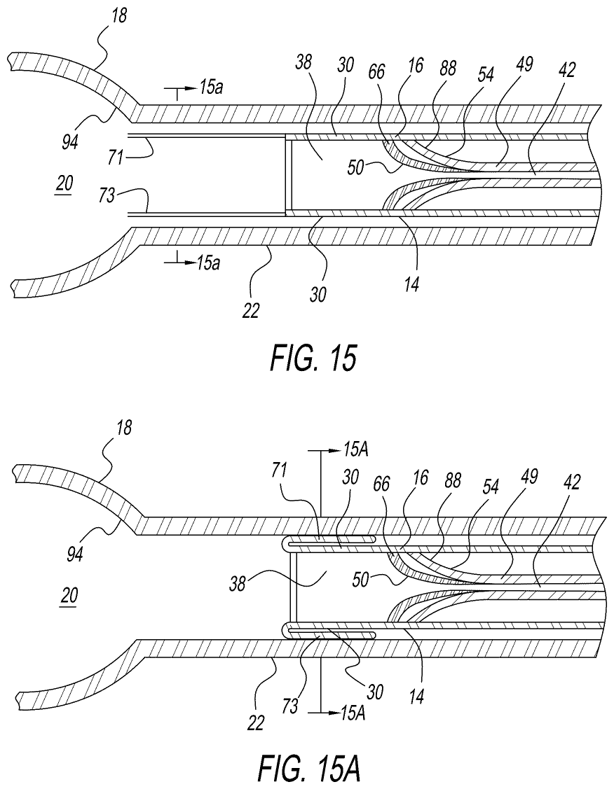

Turning now to FIG. 15, a sectional view similar to FIG. 13 is shown, illustrating a catheter having an alternative configuration. In particular, the shape and configuration of the distal ends of the catheter sheath comprise a pair of flexible flap like members that extend outwardly rom the end of the sheath

FIG. 15A is a sectional view along lines 15A-15A showing that the flap like members are two members. Each of them extends about 180 degrees or so around the circumference of the sheath.

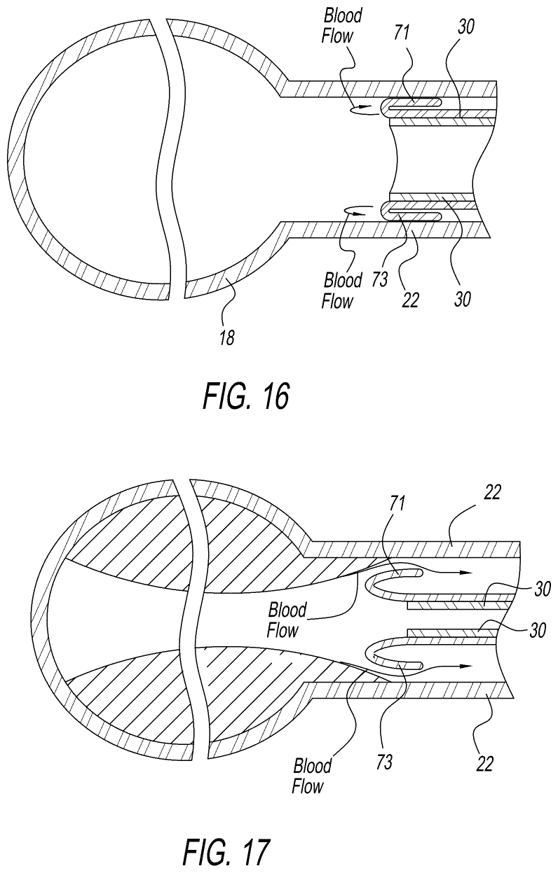

FIG. 16 illustrates the heart muscle 18, flaps 71, 73, and aorta 22. Sheath 30 is also shown. The arrows indicate the direction of blood flow.

When the heart is in its diastolic phase, the heart is relaxing. This would cause the blood that is in the aorta to want to flow back from the aorta and into the heart chamber (ventricle). As you will notice, the flaps are pulled radially outwardly by the returning blood action, acting like a parachute that captured the blood attempting to flow back into the heart muscle, so that they move radially outwardly into the space between the sheath and the inner wall of the aorta, to thereby block blood from flowing from the aorta back into the heart chamber. The flaps 71, and 73 will serve as a temporary heart valve until a new permanent prosthetic or bioprosthetic valve is placed. The presence of the flaps will allow the resection of the degenerative aortic valve 12 and its leaflets 14, and 16 without the need to use a heart lung bypass machine.

In FIG. 17 the heart is shown in the systolic phase, where the heart is pumping blood into the aorta. In this phase, the contraction of the heart squeezes blood to exert pressure on the flow of blood from the chamber downstream through the aorta.

This downstream pressure overcomes the resiliency or strength of the flaps 71, 73. This allows blood to flow past the flaps and into the space between the sheath 30 and the aorta, and then downstream into the circulatory system of the patient. By blocking the flow of blood from the aorta and into the ventricle, it may be possible to perform the valve resection without placing the patient on a heart bypass machine during the surgery.

The steps that will include resection of the defective valve, and the coupling of the new second-generation valve to the aortic anulus 94 will be performed during a rapid pacing run in which the heart is made to beat at the rate of 160-200 beats per minutes to reduce the motion of the heart during those critical brief moments.

Having described the invention in detail with reference to certain preferred embodiments, it will be appreciated that modifications and variations can exist within the spirit, which are limited only by the language of the claims.

What is claimed is:

1. A heart catheter for removing at least one leaflet of a defective heart valve comprising:
a catheter sheath having a proximal end, a distal end, and an interior passageway extending between the distal end and the proximal end;
a catheter tube insertable into the passageway of the catheter sheath, the catheter wire having a proximal end and a distal end;
a first basket coupled to the distal end of the catheter wire, and sized and configured for being received interiorly of a heart valve; and
a second basket coupled to the distal end of the catheter control tube, and being sized and configured to interiorly receive one or more valve leaflets, and the first basket.

2. The heart catheter of claim 1, wherein the first and second baskets are moveable with respect to each other along the catheter wire between a disengaged position wherein the first and second baskets are not nested together; and an engaged position wherein the first and second baskets are nested together.

3. The heart catheter of claim 2, wherein the first basket has an interior surface and an exterior surface, and the second basket has an exterior surface and an interior surface.

4. The heart catheter of claim 3, wherein the first basket is configured to be received interiorly of the heart valve, and the second and first baskets are moveable with respect to each other to capture at least one leaflet of the heart valve between the exterior surface of the first basket and the interior surface of the second basket.

5. The heart catheter of claim 4, wherein one or both of the first basket and second includes electrically conductive material capable of severing a defective heart valve leaflet from a heart.

6. The heart catheter of claim 5, wherein at least a portion of the exterior surface of the first basket contains the electrically conductive, impermeable material, and wherein the interior surface of the first basket includes a non-conductive coating.

7. The heart catheter of claim 1, wherein a shape and a size of the first basket and the second basket are substantially the same.

8. The heart catheter of claim 1, wherein a distal rim of the first and second basket is made from a shape memory material.

9. The heart catheter of claim 1, wherein a distal rim of each of the first and second basket is made from a shape memory material.

10. The heart catheter of claim 2, wherein magnets are positioned on a distal end of the first basket and a distal end of the second basket for maintaining the engaged position.

11. The heart catheter of claim 1, wherein the first basket includes one or more generally axially extending support members that provide structural rigidity while still permitting the first basket to have the flexibility to collapse so that it may fit inside the smaller diameter passageway of the catheter sheath

12. The heart catheter of claim 1, wherein the first basket includes a mesh screen.

13. The heart catheter of claim 12, wherein the mesh screen is an electrically conductive material.

14. The heart catheter of claim 12, wherein an electrically insulating coating is placed over the surface of an interior of the mesh screen.

15. The heart catheter of claim 1, wherein the shapes of the first basket and the second basket can be conical, frusto-conical, pyramidal, or frusto-pyramidal.

16. The heart catheter of claim 1, wherein the distal ends of the first basket and the second basket may be formed of two or more arch shaped segments, to match the shape of the aortic valve.

17. The heart catheter of claim 1, further including a pair of flexile flap like members that extend outwardly from the distal end of the sheath.

18. The heart catheter of claim 17 wherein the flexible flap members each extend at least about 180° around a rim of one or both of the first and second baskets.

19. The heart catheter of claim 18 wherein the flexible flaps are configured for reducing flow of blood from a blood vessel into a heart chamber during a diastolic cycle.
